(19) United States
(12) Patent Application Publication
(10) Pub. No.: US 2007/0224215 A1
(43) Pub. Date: Sep. 27, 2007

(54) Brown seaweed extracts in capsules consisting of broad-spectrum freeze dried antioxidant concentrates, Fucoidan, crystalloidals, Mangosteen, hyaluronic acid, alpha lipoic acid, green tea extract, vitamin C, and black pepper extract with delivery systems for utilization of nutrients

(76) Inventor: Kevin Buckman, Stockton, CA (US)

Correspondence Address: Kevin Buckman, 5937 Cumberland, Stockton, CA, 95219, US

(21) Appl. No.: 11/385,282

(22) Filed: Mar. 22, 2006

(51) Int. Cl. A61K 9/48; A61K 36/02

(52) U.S. Cl. 424/195.17; 424/451

(57) ABSTRACT

Method of making a Fucoidan Brown seaweed capsule with one or more or more of the following ingredients: freeze dried white and concord grape polyphenol carotenoid concentrate, mangosteen, freeze dried blueberry polyphenol carotenoid concentrate, freeze dried brown seaweed brown seaweed fucoidan carotenoid concentrate, black pepper extract, xyonix crystalloidal matrix, DMAE, ascorbic acid, green tea extraction, astaxanthin in concentrated beadlet form, Fucoidan in angstrom size and use in an ionic crystalloidal delivery system, and alpha lipoid acid.

### BACKGROUND AND SUMMARY OF THE INVENTION

This formula uses concentrated freeze dried broad-spectrum carotenoid antioxidants that are used in a capsule form to preserve the quality and effectiveness of the product. Bioavailability of a nutraceutical product is needed in order for the body to utilize the nutrients and obtain the benefits. Bioavailability of the key ingredients is included in this patent. The ingredients and delivery systems for bioavailability can be used in this patent in a capsule, gel tab, tablet, powder or beadlet form.

The active ingredients in brown seaweeds have Fucoidan and other nutrients that are in the capsule. The Fucoidan extract is produced from the brown seaweed and other seaweeds to make this capsule along with other synergistic nutrients to enhance the antioxidant effect and benefits of the brown seaweed capsule. The Fucoidan group of antioxidants possess a strong anti-oxidant effect. The addition of other antioxidants such as mangosteen, that enhances the spectrum and effects of the formula. The ionic delivery system enhances the effects, benefits, absorption, and use of these nutrients. The capsule allows for easy ingestion of formulas, and an ionized Fucoidan formula by conversion to angstrom size and placed in an ionic crystalloidal system adds to the benefits and use of this formula. The addition of other carotenoids such as white and concord grape polyphenol carotenoids concentrate, blueberry polyphenol carotenoids concentrate, brown seaweed brown seaweed carotenoids, astaxanthin carotenoid concentrate, and chlorogenic acid enhances the broad-spectrum anti-oxidant effect and wellness effects of the orally ingested capsular formula. The alkalionic (alkaline ionic) crystalloidals are important as part of the ionic delivery system and cell health. Broad spectrum antioxidants are needed to provide free radical processing and elimination. Free radicals are known to be a health hazard.

1. Method of formulation of brown seaweed capsules with broad-spectrum antioxidants and an enhanced delivery system, and

2. Use of angstrom sized Fucoidan molecules as part of the formula, with Fucoidan that is used in a powder form, from the extract of the brown seaweed of different sources such as limu, wakame, mozuku, kombu, hijiki, and other sources, and

3. Use of a freeze dried concentrated antioxidants such as blueberry carotenoids power, and brown seaweed Fucoidan-caroteinoid concentrate for broad-spectrum antioxidant effects in the formula in an enhanced delivery system in a capsule, and

4. Method for brown seaweed capsules with and without iridium to enhance the photonic and ionization effect of this capsule formula, and

5. Method of making a Fucoidan product at less cost with a delivery system in a capsule form that allows for better absorption of Fucoidan molecules, increase the metabolic and anti-oxidant effect of Fucoidan, and use of less Fucoidan extracts, and

6. Method of use of the Fucoidan product with or without a liposomal delivery system for oral capsule form, and

7. Addition of beadlet astaxanthin carotenoid concentrate for broad-spectrum antioxidant effect in the capsule, and

8. Addition into the capsules of xyonix crystalloidal matrix for better ionic delivery and cell health, and

9. The capsule formula may also consist of one or more of the following: hyaluronic acid, DMAE, ascorbic acid, black pepper extract, freeze dried white and concord grape polyphenol carotenoid concentrate, mangosteen, freeze dried blueberry polyphenol carotenoid concentrate, freeze dried brown seaweed brown seaweed Fucoidan-carotenoid concentrate, black pepper extract, xyonix crystalloidal matrix, DMAE, ascorbic acid, green tea extraction, astaxanthin in concentrated beadlet form, and

10. The formula may consist of one or more of the following antioxidants to provide a broad-spectrum range of antioxidant effects: freeze dried white and concord grape polyphenol caroteinoid concentrate, freeze dried blueberry polyphenol caroteinoid concentrate, astaxanthin caroteinoid concentrate in a powder form, in a powder form for use in a capsule, and

11. Method of using the above nutrients in a capsule, gel tab, tablet, or powder or beadlet form.
